(19) United States
(12) Patent Application Publication
(10) Pub. No.: US 2008/0095806 A1
(43) Pub. Date: Apr. 24, 2008

(54) Composition And Method For Treating Inflammatory Diseases Using Protease Inhibitors

(76) Inventors: Ian C. Bathurst, Alameda, CA (US); Philip A. Pemberton, Alameda, CA (US); David J. Sundin, Alameda, CA (US); James W. Mayhew, Petaluma, CA (US); Arturo J. Angel, Petaluma, CA (US); Philip J. Barr, Alameda, CA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/535,609

(22) PCT Filed: Nov. 20, 2003
(86) PCT No.: PCT/GB03/05049

§ 371 Date: Nov. 2, 2007

Related U.S. Application Data
(60) Provisional application No. 60/427,702, filed on Nov. 20, 2002.

(51) Int. Cl. A61K 38/55; A61K 9/00

(52) U.S. Cl. 424/400; 514/12

(57) ABSTRACT

Compositions containing a protease inhibitor and methods of use and production are described. The compositions contain an effective amount of a protease inhibitor in a carrier or diluent and are used for the treatment of inflammatory or hyperproliferic mucocutaneous disorders. The carrier or diluent is preferably a gelling agent, and the composition is a topical gel formulation containing alpha 1-antitrypsin in an aqueous liquid or viscous gel formulation.

### TECHNICAL FIELD

This invention relates to a protease inhibitor composition and use of the composition for therapeutic applications. In particular, the invention relates to a protease inhibitor composition for the treatment or prevention of hyperproliferative, inflammatory mucocutaneous disorders.

### BACKGROUND OF THE INVENTION

Hyperproliferative and inflammatory skin or mucocutaneous disorders affect millions of individuals in the United States every year. Such disorders range from mild to life threatening and include, for example, skin cancer, atopic dermatitis, psoriasis, and asthma due to the inflammation of the lung mucosa. In addition, extrinsic skin aging can be caused by chronic inflammation and insufficient skin repair due to repetitive exposures to environmental insults, e.g. ultraviolet radiation.

Atopic dermatitis is very common in all parts of the world. This chronically relapsing inflammatory skin disorder affects about ten percent of infants and three percent of the U.S. population overall. The disease can occur at any age, but is most common in infants to young adults (see, Hanifin, et al, Arch. Dermatol, 135(12):1551 (1999)). The face is often affected first, then the hands and feet. Sometimes dry red patches appear all over the body. In older children, the skin folds are most often affected, especially the elbow creases and behind the knees. In adults the face and hands are more likely to be involved.

Eczema is another example of a common inflammatory disorder. Eczema is a red, itchy, noncontagious inflammation of the skin that may be acute or chronic, with red skin patches, pimples, crusts, or scabs occurring either alone or in combination. The skin may be dry, or it may discharge a watery fluid, resulting in an itching or burning sensation. The affected skin may become infected. The various causes of eczematous dermatitis are classified as either external (irritations, allergic reactions, exposure to certain microorganisms or chemicals, etc.), congenital (inherited predisposition) and environmental (stress, heat, etc). Eczema may clear for years, only to reappear later at a different site. Eczema can come in any of several forms, including, most commonly, atopic dermatitis.

A condition similar to atopic dermatitis, but which affects mucosal tissues rather than the skin, is asthma. Asthma is a chronic lung disease characterized by inflammation of the air passages. This condition is estimated to affect about 15 million Americans and can be severe and result in death if not treated. A number of factors can exacerbate asthma including, e.g., rapid changes in temperature or humidity, allergies, upper respiratory infections, exercise, or stress. Typical treatments include bronchodilators which are given orally or delivered as an aerosol (inhaled) and, for the most difficult cases, corticosteroids. Another example of a mucocutaneous inflammatory disorder is allergic rhinitis (hay fever). Allergic rhinitis is caused by a nasal inflammation in response to an irritant or an allergen. This condition can be seasonal or occur throughout the year (perennial). Currently, allergic rhinitis is treated by the administration of antihistamines either orally or locally (i.e., using nasal sprays).

Other examples of mucocutaneous inflammatory disorders include those that involve comification and papulosquamous disorders. Examples of such disorders include lamellar ichthyosis, acne, rosacea, psoriasis, and lichen planus. Papulosquamous disorders are those characterized, as the name suggests, by scaly papules and plaques. Some of the more common papulosquamous disorders include psoriasis and lichen planus, both of which are, manifested by a local inflammation of either the skin or a mucosal tissue (e.g., in the case of oral lichen planus).

Psoriasis is a persistent skin disease. The skin becomes inflamed, producing red, thickened areas with silvery scales, most often on the scalp, elbows, knees, and lower back. Severe psoriasis may cover large areas of the body. Psoriasis is not contagious, and has some genetic basis as it is more likely to occur in people whose family members have it. In the United States about 2% of adults have psoriasis (four to five million people). Approximately 150,000 new cases occur each year. The cause of psoriasis is unknown, however, recent discoveries point to an abnormality in the functioning of key white cells in the blood stream triggering inflammation in the skin. Psoriasis is thus thought to be due, at least in part, to an abnormal immune reaction against some component of the skin. This leads to the local infiltration of inflammatory cells, including leukocytes, into the tissues, expression of cell adhesion molecules, and the up-regulation of inflammatory cytokines and growth factors. As a result, the two hallmark features of psoriasis are local inflammation and epidermal hyperproliferation. The combination of hyperproliferation with incomplete terminal differentiation leads to the formation of a thickened stratum corneum or plaques.

Hyperproliferative skin disorders result from the loss of the regulatory mechanisms that control the proliferation and differentiation of skin cells. Basal and squamous cell carcinomas are the most common forms of skin cancer. About 1.3 million cases of skin carcinomas are found in the United States per year. Both basal and squamous cell carcinoma (i.e. Kaposi's sarcoma) affect the most external layer of the skin, the epidermis, and begin at the basal cell layer and at the upper cell layer of the epidermis, respectively. Although these skin carcinomas are slow growing and usually benign, they can, if not treated, grow and invade other tissues.

In addition to changes resulting from inflammatory and hyperproliferative disorders, the appearance and characteristics of the skin also change as the body ages. Chronologically aged (intrinsically aged) mucocutaneous surfaces show a slight atrophy of the epidermis and weakening the dermal/epidermal junction. Dryness of the skin is a common phenomenon. The dermis shows a decrease in cell numbers and elastic fibers and thus, a reduction in skin elasticity. Capillaries are also fragile as evidenced by bruisability. Collagen metabolism is slower, and there is a progressive lowering in concentration of glycosaminoglycans, thus sagging of the skin occurs.

There is a decreased ability to mount inflammatory responses in aged skin and an increase in the time of healing after injury. Aging is accelerated in those areas exposed to environmental insults, such as, i.e., irritating substances and sunlight (ultraviolet radiation), due to the development of local skin inflammation. The skin aging process resulting from exposure to sunlight is known as “photoaging.” Photoaging accounts for about 80% of the visible changes of skin aging. It induces deep wrinkles not erased by stretching, pigmentary alterations with areas of hyper- and hypo-pigmentation (actinic lentigines and leukodermas), and a variety of benign, premalignant, and malignant neoplasms. The dermis shows evidence of chronic inflammation with increased cellularity and enlarged fibroblasts.

While certain treatments have been developed for some of these conditions, the treatments are often ineffective, not tolerated by certain individuals, or associated with one or more side effects that limit their use. With some of these conditions, no effective treatments currently exist. The side effects exhibited by currently available treatments include a rebound of the disease activity upon withdrawal of medication (i.e., glucocorticoids, cyclosporin A-like drugs), an increase in the incidence of cancer (i.e., PUVA), and toxicity (i.e., antimetabolites, such as methotrexate). Additionally, certain procedures are extremely inconvenient (i.e., coal tar treatments) or invasive (i.e., surgery). Current treatments for skin photodamage include retinoids and alpha-hydroxy-acids that exhibit light sensitivity, limited efficacy, and untoward side effects. Excipients that are safe and contain active ingredients are rare and in high demand by the cosmeceutical industry.

Currently, there is a largely unmet medical need for effective and safe compositions for the treatment of inflammatory mucocutaneous disease or disorders. Topical formulations are typically preferred over the orally delivered drug in order to avoid adverse systemic side effects. Nevertheless, developing a suitable topical formulation for clinical use in the treatment of inflammatory mucocutaneous disease or disorders poses considerable challenges. Particularly, problems associated with sufficient penetration through the skin or mucous membrane remain a concern.

Additionally, in topical formulations containing a gel or gelling agent, problems with pH, solubility, consistency, and sterility of the gel arise. Achieving a uniform pH in a gel formulation is required for safety in topical products but is difficult to obtain. Furthermore, achieving sterility of a gel formulation may be difficult if filter sterilization and heat sterilization techniques cannot be used without destroying the activity of the active ingredient. In addition, suitable topical gels must have a solubility and consistency that allow spreading the gel over the area to be treated without losing contact with the site of injury.

In view of the foregoing, it is readily apparent that there is a great need in the art for new protease inhibitor composition for effective treatments of a large number of inflammatory and hyperproliferative mucocutaneous disorders. The present invention addresses these and other needs.

### SUMMARY OF THE INVENTION

A protease inhibitor composition and methods of making and using the composition are described herein. The composition is a pharmaceutical composition containing an effective amount of a protease inhibitor in a pharmaceutically acceptable carrier or diluent. The composition is useful for preventing and treating a variety of hyperproliferative and inflammatory mucocutaneous disorders.

Preferably, the protease inhibitor in the composition described herein is a serine protease inhibitor. Most preferably, the protease inhibitor is an alpha 1-antitrypsin. The preferred carrier is a gelling agent, optionally in combination with a physiological buffer. The preferred concentrations of protease inhibitor in the composition is from about 0.001% to about 30% w/w, more preferably from about 0.1% to about 3% w/w, or most preferably from about 1% to about 1.5% w/w.

The alpha 1-antitrypsin in the composition is a natural protein, isolated protein, synthetic protein, recombinant protein, modified protein, biologically active fragment, substantially homologous protein, oligopeptide, homodimer, heterodimer, variants of the protein, derivative, analog, fusion protein, or agonist, of alpha 1-antitrypsin.

The physiological buffer in the pharmaceutical composition described herein includes buffers such as tris, histidine, triethanolamine, and salts. Salts are preferably NaCl, KCl, or phosphate salts. The physiological buffer preferably contains 0-250 mM phosphate, 0-250 mM NaCl, or 0-250 mM KCl. More preferably, the physiological buffer contains 5-100 mM phosphate, 5-100 mM NaCl, or 5-100 mM KCl. The pH range of the physiological buffer used in the pharmaceutical composition is preferably from about pH 6 to about 9, more preferably from about pH 6.5 to 8, and most preferably about pH 7 to about 7.5.

The gelling agent in the pharmaceutical composition described herein includes any pharmaceutically suitable gelling agents such as, for example, hydroxyethyl cellulose, hydroxypropyl cellulose, polyacrylic acid, polyoxyethylene-polyoxypropylene block copolymer, or a combination thereof. The concentration range of the gelling agent is preferably from about 0.1% to about 50% w/w, more preferably from about 0.5% to about 5% w/w, or most preferably from about 0.25% to about 2% w/w.

The composition optionally contains preservatives, reducing agents (i.e., dithiothreitol, N-acetylcysteine, cysteine, glutathione), antioxidants (i.e., ascorbic acid, methionine), chelating agents (i.e., EDTA, or citrate), bulling agents/stabilizers (i.e., trehalose, sucrose, glycine, mannitol, dextrans, sorbitol glycerol, propylene glycol, albumin, disaccharides such as sucrose, cyclic oligosaccharides such as cyclodextrins, L-ascorbic acid or its derivatives, tocopherol, or a combination thereof, among others), surfactants (i.e., tween, nonidet triton, or span), excipients, or combinations thereof.

Preservatives are used to maintain integrity of the composition and include, for example, quaternium, methylparaben, phenol, para-hydroxybenzoate compounds, propyleneglycol, propylparaben, or a combination thereof, at suitable concentrations, for example, from about 0.001% to about 0.5% w/w.

In accordance with the method for treating or preventing a variety of inflammatory or hyperproliferative mucocutaneous diseases, disorders, or syndromes, including, for example, dermatologic disorders, disorders of the lung, disorders of the ear, ocular disorders, disorders of the gastrointestinal tract, and disorders of the urinary tract.

In accordance with the method for making the composition, the protease inhibitor is combined with the pharmaceutically acceptable carrier or diluent, preferably a gelling agent. The composition is prepared according to a procedure that ensures suitable pH conditions within the gel, optimum protease inhibitor solubility, gel consistency, stability, and sterility in the resulting composition.

In another aspect of the invention, there is provided a pharmaceutical pack or kit for treating and/or preventing hyperproliferative and inflammatory mucocutaneous disorders including one or more containers filled with one or more of the ingredients of the pharmaceutical compositions described herein, and instructions for use thereof. Optionally associated with such container can be a notice in the form prescribed by a governmental agency regulating the manufacture, use or sale of pharmaceuticals or biological products, which notice reflects approval by the agency of manufacture, use or sale for human administration.

### BRIEF DESCRIPTION OF THE FIGURES

FIG. 1 is a graph showing percent penetration versus time for nine formulations of the alpha 1-antitrypsin composition described herein. The cumulative transdermal absorption of alpha 1-antitrypsin is presented as the percent of the applied dose recovered in the reservoir at each time point. The skin specimens that showed ≧1.0% absorption in the reservoir compartment were removed from the reservoir data for the final data analysis due to apparent defect in barrier performance.

FIG. 2 is a bar graph showing percent recovery for nine formulations of the alpha 1-antitrypsin composition described herein. The localization of alpha 1-antitrypsin in the various skin compartments is presented as percent of the applied dose (DPM) recovered in each compartment.

### DETAILED DESCRIPTION OF THE INVENTION

A composition containing a protease inhibitor and a method for preventing or treating hyperproliferative, inflammatory mucocutaneous disorders and a method for making the composition are provided herein. The composition contains a protease inhibitor in a pharmaceutically acceptable carrier or diluent. The preferred protease inhibitor is a serine protease. The preferred carrier or diluent is a gelling agent. Gel formulations containing an effective amount of the protease inhibitor have been found to exhibit an unexpectedly superior activity and stability for the use as a therapeutic composition. The gel formulations described herein maintain a uniform pH within a desired pH range and have demonstrated improved protease inhibitor solubility, gel consistency, and sterility.

### Protease Inhibitor

The composition described herein contains a protease inhibitor that is effective in treating inflammatory conditions. Protease inhibitors comprise approximately 10% of the human plasma proteins. A large number of naturally occurring protease inhibitors serve to control endogenous proteases by limiting their reactions locally and temporally. Tissues that are particularly prone to proteolytic attack and infection, i.e. those of the respiratory tract, are rich in protease inhibitors. Endogenous proteolytic enzymes serve to degrade invading organisms, antigen-antibody complexes and certain tissue proteins that are no longer necessary or useful. In a normally functioning body, proteolytic enzymes are produced in a limited quantity and are regulated in part through the synthesis of protease inhibitors.

A disturbance of the protease/protease inhibitor balance can lead to protease-mediated tissue destruction, including emphysema arthritis, glomerulonephritis, periodontitis, muscular dystrophy, tumor invasion and various other pathological conditions. In certain situations, i.e. in severe pathological processes such as sepsis or acute leukemia, the amount of free proteolytic enzymes increases due to the release of enzymes from the secretory cells. In addition, a diminished regulating inhibitor capacity of the organism may also cause alterations in the protease/protease inhibitor balance. An example of such a diminished regulating inhibitor capacity is alpha 1-antitrypsin deficiency, which is highly correlated with the development of pulmonary emphysema.

The protease inhibitor in the composition described herein exhibits anti-inflammatory activity, and is preferably provided as an isolated and substantially purified compound in the composition. Protease inhibitors useful in the composition described herein generally include, but are not limited to, aspartyl protease inhibitors, cysteine protease inhibitors, metalloprotease inhibitors, serine protease inhibitors, alpha 1-antitrypsin, alpha 1-antichymotrypsin, secretory leukocyte protease inhibitor, C-reactive protein, serum amyloid A protein, alpha 2-macroglobulin, eglin, elasnin 3, elastinal, aprotinin, leupepsin, antipain, pepstatin, phosphoramidon, trypsin inhibitors from albumin or soy beans, gabaxate mesylate, or a combination thereof, among others. The protease inhibitor is preferably a serine protease inhibitor. More preferably, the protease inhibitor is an alpha 1-antitrypsin or a recombinant alpha 1-antitrypsin (rAAT).

The protease inhibitor in the composition can be derived from many species and includes natural, synthetic, or recombinant forms of the protein. The protease inhibitor in the composition further includes peptide fragments, biologically active fragments, substantially homologous polypeptides, oligopeptide, homodimers, heterodimers, variants of the polypeptides, modified polypeptides, derivatives, analogs, fusion proteins, or agonists of the polypeptide of a protease inhibitor. The protease inhibitor also includes small synthetic molecules that can inhibit proteases.

The protease inhibitor in the composition is either isolated, synthesized or produced recombinant technology. Natural protease inhibitors are isolated from biological fluids including, for example, semen, blood, urine, plasma (i.e., autologous, or homologous human plasma), milk, or cultured eukaryotic (insect or mammalian cells) or prokaryotic cells (yeast, bacteria) by techniques known to those skilled in the art. Alternatively, protease inhibitors can also be produced by recombinant DNA methods. Such methods are well known to those of ordinary skill in the art. One example of a method of producing the protease inhibitor by using recombinant DNA techniques entails the steps of (1) identifying and purifying the protease inhibitor polypeptide of interest, (2) determining the N-terminal amino acid sequence of the purified polypeptide, (3) synthetically generating a DNA oligonucleotide probe that corresponds to the N-terminal amino acid sequence, (4) generating a DNA gene bank from human or other mammalian DNA, (5) probing the gene bank with the DNA oligonucleotide probe, (6) selecting clones that hybridize to the oligonucleotide, (7) isolating the inhibitor gene from the clone, (8) inserting the gene into an appropriate vector such as an expression vector, (9) inserting the gene-containing vector into a microorganism or other expression system capable of expressing the inhibitor gene, and (10) isolating the recombinantly produced protease inhibitor. The above techniques are more fully described in laboratory manuals such as “Molecular Cloning: A Laboratory Manual” Latest Edition by Sambrook et al., Cold Spring Harbor Press (1989). Protease inhibitors are also be produced in recombinant eukaryotic or prokaryotic expression systems, and purified with column chromatography. Useful expression systems include, but are not limited to, E. coli, insect, or yeast expression systems.

Yet another method of producing protease inhibitors, or biologically active fragments thereof, is by peptide synthesis. For example, once a biologically active fragment of protease inhibitors is found, it can be sequenced, for example by automated peptide sequencing methods. Alternatively, once the gene or DNA sequence which codes for the protease inhibitor is isolated, for example by the methods described above, the DNA sequence can be determined, which in turn provides information regarding the amino acid sequence. Thus, if the biologically active fragment is generated by specific methods, such as tryptic digests, or if the fragment is N-terminal sequenced, the remaining amino acid sequence can be determined from the corresponding DNA sequence.

Once the amino acid sequence of the peptide is known, for example the N-terminal 20 amino acids, the fragment can be synthesized by techniques well known in the art, as exemplified by “Solid Phase Peptide Synthesis: A Practical Approach,” E. Atherton and R. C. Sheppard, IRL Press, Oxford England. Similarly, multiple fragments can be synthesized which are subsequently linked together to form larger fragments. These synthetic peptide fragments can also be made with amino acid substitutions at specific locations in order to test for agonistic and antagonistic activity in vitro and in vivo.

As used herein, the term “biologically active fragment of a protease inhibitor” refers to fragments exhibiting activity similar, but not necessarily identical, to the activity of one or more of the protease inhibitors described herein. The biologically active fragments may exhibit an increase or improvement in a desired activity and/or a decrease in an undesirable activity.

### Modified Protease Inhibitor

Protease inhibitors useful in the composition include polypeptides encompassing a variety of modifications, particularly those that are present in polypeptides synthesized by expressing a polynucleotide in a host cell.

It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, or by chemical modification techniques. Modifications which may be present in polypeptides employed herein include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, anchor formation, hydroxylation, iodination, methylation, myristoylation, oxidation, pegylation, proteolytic processing, phosphorylation, prenylation, racemization, selenoylation, sulfation, transfer RNA mediated addition of amino acids to proteins such as arginylation and ubiquitination, or a combination thereof, among others.

It will be appreciated that polypeptides are not always entirely linear. For instance, polypeptides may be branched as a result of ubiquitination, and they may be circular, with or without branching, generally as a result of post-translational events, including natural processing events. Circular, branched and branched circular polypeptides may be synthesized by non-translational natural processes and by synthetic methods, as well.

Modifications occur anywhere in a polypeptide, including the peptide backbone, the amino acid side chains and the amino or carboxyl termini. Blockage of the amino or carboxyl group in a polypeptide by a covalent modification occurs in natural or synthetic polypeptides and such modifications are present in polypeptides of the present invention, as well. In general, the nature and extent of the modifications are determined by the host cell's post-translational modification capacity and the modification signals present in the polypeptide amino acid sequence. It will be appreciated that the same type of modification may be present in the same or varying degrees at several sites in a polypeptide. Also, a polypeptide may contain more than one type of modifications.

Variants of a protease inhibitor include polypeptides that differ in amino acid sequence from a reference polypeptide. Generally, differences are limited so that the sequences of the reference and the variant are closely similar overall and, in many regions, identical. A variant and reference protease inhibitor may differ in amino acid sequence by one or more substitutions, additions, deletions, fusions and truncations, which may be present in any combination.

The protease inhibitor described herein may include truncated and/or N-terminally or C-terminally extended forms of the polypeptide, analogs having amino acid substitutions, additions and/or deletions, allelic variants and derivatives of the polypeptide, so long as their sequences are substantially homologous to the native protease inhibitor polypeptide.

Specifically, as will be appreciated by those skilled in the art, the protease inhibitor in the composition described herein includes polypeptides having slight variations in amino acid sequences or other properties. Such variations may arise naturally as allelic variations, as disclosed above, due to genetic polymorphism, for example, or may be produced by human intervention. (i.e., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecule. Substitutions may be designed based on, for example, the model of Dayhoff, et. al., Atlas of Protein Sequence and Structure, Nat'l Biomed. Res. Found., Washington, D.C. (1978). These modifications can result in changes in the amino acid sequence, provide silent mutations, modify a restriction site, or provide other specific mutations.

As one of skill in the art will appreciate, and as discussed above, the protease inhibitor of the composition can be fused to a heterologous polypeptide sequence. For example, the protease inhibitor (including fragments or variants thereof) may be fused to one or more additional protease inhibitors, or other anti-inflammatory peptides. The protease inhibitor may also encompass genetically engineered soluble fusion proteins comprised of a protease inhibitor polypeptide, for example, alpha 1-antitrypsin and various portions of other proteins such as, for example, a membrane-bound protein.

### Pharmaceutical Composition

The pharmaceutical composition provided herein contains a protease inhibitor, in an amount effective for the treatment or prevention of inflammatory mucocutaneous or respiratory diseases, and a pharmaceutically acceptable carrier or diluent. As mentioned above, the preferred protease inhibitor is a serine protease, such as alpha 1-antitrypsin. The preferred carrier is a gelling agent. The term “pharmaceutically acceptable” is interpreted herein to mean that the substance is approved by a regulatory, agency of the Federal or a state government or listed in the U.S. Pharmacopoeia or other generally recognized pharmacopoeia for use in animals, and more particularly in humans. The protease inhibitor is optionally in combination with one or more other pharmaceutically active agents.

The protease inhibitor can be formulated as a neutral substance (free base) or in the salt form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, or procaine, among others.

A pharmaceutically effective amount of the protease inhibitor in the composition is, for example, within the range of from about 0.001% to about 30% w/w, preferably from about 0.01% to about 10% w/w, more preferably from about 0.05% to about 5% w/w, from about 0.1% to about 3% w/w, or from about 0.5% to about 2.5% w/w, and most preferably from about 1% to about 1.5% w/w.

The term “carrier” as used herein refers to a diluent, adjuvant, excipient, or vehicle in which the therapeutic agent is administered. Such pharmaceutical carriers include, for example, starch, glucose, lactose, sucrose, gelatin, malt, rice, flour, chalk, silica gel, sodium stearate, glycerol monostearate, talc, sodium chloride, dried skim milk, glycerol propylene, glycol water, ethanol, oil (i.e., oil derived from petroleum, animals, plants including peanut oil, sunflower, oil, olive oil, almond oil, walnut oil, soybean oil, pine oil, sesame oil), among others. Water is a preferred carrier when the pharmaceutical composition is administered topically and by injection. Saline solutions and aqueous dextrose and glycerol solutions are also employed as liquid carriers, particularly for injectable solutions and topical applications. In a preferred embodiment, the pharmaceutical composition is a gel formulation designed for topical administration or administration to a mucous membrane and includes a pharmaceutically effective amount of a protease inhibitor, a gelling agent and, optionally, a physiological buffer. The composition also optionally contains water.

The preferred pharmaceutical composition is a protease inhibitor gel formulation that contains alpha 1-antitrypsin, a physiological buffer, and a gelling agent. The most appropriate physiological buffer for combination with the gelling agent includes tris, histidine, trietholamine, and salts. The salts are preferably sodium chloride, potassium chloride or phosphate salts. In a preferred formulation, the physiological buffer contains 0-250 mM phosphate, 0-250 mM NaCl, and 0-250 mM KCl. In a more preferred formulation, the physiological buffer contains 5-100 mM phosphate, 5-100 mM NaCl; and 5-100 mM KCl. The pH range of the physiological buffer used in the pharmaceutical compositions of the invention is preferably within the range of from about pH 6.0 to about 9.0, more preferably between about pH 6.5 to about 8.0, and most preferably between about pH 7.0 to about 7.5.

The gelling agent of the pharmaceutical composition includes any pharmaceutically suitable gelling agents such as, for example, hydroxyethyl celluloses, poloxamers, hydroxypropyl celluloses (HPC), polyacrylic acids, polyoxyethylene-polyoxypropylene block copolymers, or any combination thereof. The concentration range of the gelling agent in the composition is, for example, from about 0.1% to about 50% w/w, preferably from about 0.1% to about 10% w/w, from about 0.5% to about 5% w/w, from about 1.5% to about 4% w/w, or from about 0.25% to about 2% w/w, or most preferably from about 0.3% to about 1% w/w.

The preferred gelling agent is a polyacrylic acid having a concentration from about 0.25% to about 2% w/w or from about 0.3% to about 1% w/w of the composition. The pH of the polyacrylic acid gel is, for example, within the range of from about 5.0 to about 9.0, preferably between about 6.0 to about 8.0, and more preferably between about 6.5 to about 7.4. Polyacrylic acid polymer is also known as carbomer. A preferred polyacrylic acid polymer is sold under the trademark Carbopol™ polymer (Noveon, Inc., Cleveland, Ohio). The preferred grade of Carpobol™ carbomer is P-934, or P-980.

Alternatively, the gelling agent is a polyoxyethylene-polyoxypropylene block copolymer in a concentration of from about 18% to about 35% w/w in the composition. Preferably, the concentration of the polyoxyethylene-polyoxypropylene block copolymer is from about 18% to about 25% w/w. The pH of the block copolymer gel is, for example, within the range of from about 5.0 to about 9.0, preferably between about 6.0 to about 8.0, and more preferably between about 6.5 to about 7.4. A preferred polyoxyethylene-polyoxypropylene block copolymer, also known as poloxamer, is sold under the trademark Pluronic™ (BASF, Mt. Olive, N.J.) The preferred grade of Pluronic™ Poloxamer is F-127 (poloxamer 407).

As another alternative, the gelling agent is a hygroscopic high molecular weight polymer. Hygroscopic high molecular weight polymers include, for example, cellulose derivatives such as methylcellulose, hydroxyethyl cellulose, or hydroxypropyl cellulose (HPC); anhydrous maleic acid-methylmethacrylate copolymers or esters thereof (e.g., methylesters and ethylesters); polyvinylpyrrolidine or derivatives thereof, (e.g., N-methylvinylpyrrolidine); vinyl-acetate; polyvinyl-alcohol; or a combination thereof. In a preferred embodiment, the gel contains from about 1% to about 5% w/w of a cellulose derivative, for example, hydroxypropyl cellulose (HPC). HPC has molecular weight of about 370,000 to 1,150,000 D. Preferably, the cellulose derivative is present in the composition in a concentration at about 1.50% w/w.

Within the scope of the invention described herein are pharmaceutical compositions containing a combination of the protease inhibitor and one or more additional pharmaceutically active agents. Pharmaceutically active agents useful in the composition include, without limitation, corticosteroids such as, for example, hydroxytiamcinolone, alpha methyl dexamethasone, dexamethasone acetate, betamethasone, beclomethasone dipropionate, betamethasone benzoate, betamethasone dipropionate, betamethasone valerate, clobetasol valerate, clobetasol propionate, desonide, desoxymethasone, dexamethasone, difluorosone diacetate, diflucortolone valerate, fluadrenolone, fluclorolone acetonide, flumethasone pivalate, fluocinolone acetonide, fluocinonide, flucortine butylester, flucortolone, fluprednidine (fluprednylidene) acetate, flurandrenolone, halcinonide, hydrocortisone acetate, hydrocortisone butyrate, hydrocortisone valerate, 11-desoxycortisol, methylprednisolone, triamcinolone, triamcinolone acetonide, triamciriolone diacetate, triamcinolone hexacetonide, cortisone, cortodoxone, flucetonide, fludrocortisone, difluorosone diacetate, fluradrenolene acetonide, medrysone, amcinafel, amcinafide, betamethasone and the balance of its esters, chloroprednisone, clocortelone, clocortelone pivalate, clescinolone, dichlorisone, difluprednate, flucloronide, flunisolide, fluoromethalone, fluperolone, fluprednisolone, hydrocortisone, meprednisone, paramethasone, paramethasone acetate, prednisolone, prednisolone acetate, prednisolone tebutate, prednisone, beclomethasone dipropionate, alclometasone dipropionate, mometasone furoate, or combinations thereof.

Other pharmaceutically active agents include, for example, antibiotics, cytotoxic drugs, antivirals, anti-inflammatory drugs (i.e., salicylates, colchicine, para-aminophenol, propionic acid, piroxicam, ketorolac, ketoprofen, cyclooxygenase type II inhibitors and indomethacin, among others), antihelmintics, hormones, growth factors, vitamins, antineoplastic agents, immune-response agents, immunosuppressive agents (i e., FK506, tacrolimus, pimecrolimus, among others), antithrombotics, sulfones, sunscreens, local anesthetics (i.e., proparacaine), muscle relaxants, blood regulators, anticoagulants, hemostatics, sedatives, analgesics, adrenergics, antispasmodics, bone-active agents, prostaglandins, anorexigenics, cholinergics, anticholinergics, sulfonamides, or a combination thereof, among others.

In one embodiment, the pharmaceutical composition contains an antibiotic. A preferred antiobiotic includes, macrolid antibiotics, penicillins, tetracyclins, cephalosporins, quinolones, fluoroquinolones, neomycin, gentamycin, vancomycin, or a combination thereof.

Clinically, macrolide antibiotics are used principally for treating infections with Streptococci, Staphylococci, and Pneumococci. Generally the toxicity of macrolide antibiotics is low. Esters of macrolide antibiotics have become therapeutically important because they result rapidly in higher blood levels, and further they are practically free of odor and are highly stable. Macrolide antibiotics are classified according to the size of the macrocyclic lactone ring. Macrolide antibiotics are polyfunctional molecules, most of which have at least one amine sugar and are basic.

Suitable macrolide antibiotics include those with 12-member lactone rings such as methymycin and neomethymycin. Also included are macrolide antibiotics with 14-member lactone rings, of which the preferred representatives are the erythromycins, produced from Streptomyces erythreus. Examples include, erythromycin A, erythromycin B, erythromycin C, erythromycin D, erythromycin E, erythromycin estolate, erythronolid, and clarythromycin. Other examples of macrolide antibiotics with 14-member lactone rings include, megalomycin and its derivatives, picromycin, narbomycin, oleandomycin, triacetyl-oleandomycin; and the neutral compounds laukamycin, kujimycin A, albocyclin, and cineromycin B.

Macrolide antibiotics having 16-member rings include, carbomycin (Magnamycin) and its derivatives (i.e. niddamycin), spiramycin and its derivatives, leucomycin and its derivatives (i.e. midecamycin, maridomycin, tylosin, cirramycin, and juvenimicins); and the neutral representatives chalcomycin and neutramycin. Examples of macrolide antibiotics with larger lactone rings, i.e. having 26-40 or more ring members, include pimaricin, lucensomycin, nystatin, amphotericin B, hamycin, candicidin A and B, candidin, and levorin. The effectiveness of this group is practically exclusively against fungi and yeasts.

Also within the scope of the invention is a composition containing a toxin or a cytotoxic agent, as a pharmaceutically active agent, in combination with the protease inhibitor. Such composition and method is used for specific destruction of cells (i.e., the destruction of tumor cells) by administering the protease inhibitor in association with toxins or cytotoxic prodrugs.

As used herein, the term “toxin” refers to compounds that bind and activate endogenous cytotoxic effector systems, radioisotopes, holotoxins, modified toxins, catalytic subunits of toxins, or a combination thereof, among others. Toxins that may be used according to the methods described herein include, but are not limited to, radioisotopes known in the art, compounds such as, for example, antibodies (or a complement fixing portion thereof) that bind an inherent or induced endogenous cytotoxic effector system, thymidine kinase, endonuclease, RNAse, alpha toxin, ricin, abrin, pseudomonas exotoxin A, diphtheria toxin, saporin, momordin, gelonin, pokeweed antiviral protein, alpha-sarcin, cholera toxin, or a combination thereof, among others.

As used herein, the term “cytotoxic prodrug” refers to a non-toxic compound that is converted by an enzyme, normally present in the cell, into a cytotoxic compound. Cytotoxic prodrugs that may be used in the composition described herein includes, but are not limited to, glutamyl derivatives of benzoic acid mustard alkylating agent, phosphate derivatives of mitomycin C, cytosine arabinoside, doxorubicin, and phenoxyacetamide derivatives of doxorubicin.

The pharmaceutical composition optionally contains additional compounds that assist in maintenance, storage, stabilization, cosmetic applications, or serve to prevent aggregation. These compounds include, without limitations, preservatives, reducing agents (i.e., dithiothreitol, N-acetylcysteine, 2-mercaptoethanol, cysteine, glutathione), antioxidant agents (i.e., ascorbic acid, methionine), metal chelating agents (i.e., EDTA, citrate), bulking agents/stabilizers (i.e., trehalose, glycine, mannitol, dextrans, sorbitol, glycerol, propylene glycol, albumin, disaccharides such as sucrose, cyclic oligosaccharides such as cyclodextrins, L-ascorbic acid or its derivatives, tocopherol or a combination thereof, among others), surfactants (i.e., tween, nonidet, triton, (TRITON X-114 (polyethylene glycol tertiary octylphenyl ether), TRITON X-100 (polyethylene glycol mono [p-(1,1,3,3-tetramethyl-butyl) phenyl] ether)), and/or span; dyes, excipients; perfumes, fragrances, opacifiers, or a combination thereof. Such materials, when added, should not unduly interfere with the activity of the pharmaceutically active drug or agents nor should they possess irritating properties.

Preservatives are optionally included in the composition described herein to maintain the integrity of the composition. It is known that formulations containing an aqueous phase in combination with a protein are susceptible to attack by bacteria and fungi. Microbial growth not only contaminates the formulation but is a potential toxicity hazard and a source of infection for patients. It is especially important to minimize microbial growth in topical formulations applied to broken or inflamed skin. Viscosity degradation reported with some polymers when exposed to microbial contamination is also of concern. Preservatives useful in the composition include, for example, without limitation, quaternium, methylparaben, phenol, para-hydroxybenzoate compounds, propyleneglycol, propylparaben, or a combination thereof.

Stabilizers and/or bulking agents are agents that help to preserve biological activities on a long-term basis and also improve the water solubility of a protease inhibitor. Accordingly, in a preferred embodiment, the composition contains a stabilizer/bulking agent such as, for example, albumin from about 0.01% to 5% w/w, and preferably from about 0.1% to about 1% w/w; sucrose from about 0.5% to about 30.0% w/w, and preferably from about 1.0% to about 10.0% w/w, or cyclodextrin from about 1.0% to about 10% w/w, and preferably from about 2.0% to about 5.0% w/w.

Optionally, the composition described herein additionally contains agents including, for example, an HMG-CoA reductase inhibitor. HMG-CoA reductase inhibitors suitable for use in the composition and method described herein include, but are not limited to, mevastatin, lovastatin, fluvastatin, pravastatin, simvastatin, dalvastatin, cerivastatin and atorvastatin, oxysterols, and 25-hydroxycholesterol, among others.

### Route of Administration and Dosage

In accordance with the method of treating or preventing a hyperproliferative and inflammatory mucocutaneous disorder, the composition described herein is administered to a subject in need of treatment or desiring prevention of the disorder. The subject is, for example, a mammal, including a human or an animal.

Formulations of the composition are delivered to the individual by any pharmaceutically suitable means of administration. Various delivery systems are known and can be used to administer the composition by any convenient route, for example, the composition is administered by a transdermal, intraperitoneal, intracranial, intravaginal, intrauterine, oral, rectal ophthalmic (including intravitreal or intracameral), nasal, topical (including buccal and sublingual), or parenteral route (including subcutaneous, intraperitoneal, intramuscular, intradermal, intracranial, intratracheal, and epidural).

In a preferred embodiment, the pharmaceutical composition is prepared for topical administration. The protease inhibitor, along with any other optional ingredients as set forth above, are combined in in a physiological buffer, such as saline. Preferably, the protease inhibitor is combined with one of more preservatives or antimicrobial agents commonly used in topical preparations. The solution or suspension is purified or sterilized prior to use and may be administered once or several times daily.

Most preferably, the composition is a gel formulation that is administered by swabbing, brushing or otherwise coating a site of injury or a wound with the gel. Alternatively, the composition may be administered to the skin or mucosal surface in a suppository, absorption through epithelial or mucocutaneous linings (i.e., oral mucosa, rectal, vaginal, nasal and intestinal mucosa, etc.) or eye drops. The composition is optionally prepared with a muco adhesive polymer that binds to the site of injury. Topical therapy may also be useful prophylactically in areas that are known to have a high probability of inducing an inflammatory response. In these instances the treatment may be instituted immediately to help prevent subsequent complications.

Alternatively, the composition is delivered in a controlled release system. In one embodiment, a pump may be used (see, Sefton, Biomed. Eng. 14:201 (1987)). Osmotic mini-pumps may also be used to provide controlled delivery of high concentrations of the composition through cannulae to the site of interest. In another embodiment, polymeric materials can be used (see, Ranger and Peppas, J., Macromol. Sci. Rev. Macromol Chem. 23:61 (1983); and Levy et al., Science 228:190 (1985)). In yet another embodiment, a controlled release system can be placed in proximity of the therapeutic target, i.e., the lung. Additional controlled release systems known to those skilled in the art are discussed in a review article by Langer, Science 249:1527-1533 (1990). Furthermore, the composition described herein may be incorporated into biodegradable polymers (i.e., encapsulation in liposomes, microparticles, and microcapsules, among others) allowing for sustained release of the compound. The polymers are being implanted in the area of the body where the drug delivery is desired, for example, at the site of a tumor or an injury so that the composition is slowly released systemically. Biodegradable polymers and their use are described, for example, in detail in Brem et al., J. Neurosurg. 74:441-446 (1991), which is hereby incorporated by reference in its entirety. Within other embodiments, the composition may be placed in any location such that the compound is continuously released into the aqueous humor. Administration can be systemic or local.

The composition may conveniently be presented in unit dosage form prepared by conventional pharmaceutical techniques. Such techniques include the steps of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product. In another embodiment, the composition is formulated for parenteral applications administration. Formulations suitable for parenteral administration include aqueous and non-aqueous sterile injection solutions. These solutions contain anti-oxidants, buffers, bacteriostats and solutes that render the formulation isotonic with the blood of the intended recipient. Additionally, the formulation contains aqueous and non-aqueous sterile suspensions including suspending agents, thickening agents, or both, among others. Where necessary, the composition may also include a solubilizing agent and a local anesthetic such as lignocaine to ease pain at the site of administration.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The composition may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Topical formulations may be prepared from sterile powders, granules and tablets of the kind previously described.

Alternatively, the composition is administered orally. Oral formulations include standard carriers such as pharmaceutical grades of mannitol, lactose, starch, magnesium stearate, sodium saccharine, cellulose, magnesium carbonate, sucrose, trehalose, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

The pharmaceutically effective amount of the protease inhibitor that is effective in the treatment, amelioration or prevention of a mucocutaneous or skin disease or disorder can be determined by standard clinical techniques. In addition, in vitro assays are employed to help identify optimal dosage ranges. In particular, the dosage of the protease inhibitor will depend on the disease state or condition being treated, other clinical factors such as weight and condition of the human or animal, and the route of administration of the composition. The precise dose to be employed in the formulation, therefore, should be decided according to the judgment of the practitioner and each patient's circumstances. Effective doses may be extrapolated from dose-response curves derived from in vitro or animal model test systems.

For treating humans or animals, a dosage between approximately 0.05 to 100 mg drug/kilogram of body weight is a typical broad range for administering the pharmaceutical composition. The methods provided herein contemplate single as well as multiple administrations, given either simultaneously or over an extended period of time. The individual dosage is determined according to the particular condition being treated. The compositions may be applied from one to six times or more daily or otherwise as is necessary to treat the skin condition. If a pharmaceutically active agent such as corticosteroid is used, then the composition is preferably applied one to four times daily, more preferably, once or twice daily. In the most preferred application, the composition is applied as a thin layer to the affected area twice daily and rubbed into the skin completely.

### Methods of Use of the Pharmaceutical Composition

A method of treating or preventing inflammatory or hyperproliferic mucocutaneous diseases, disorders, or syndromes is provided herein. The diseases, disorders or syndromes to be treated or prevented (collectively referred to herein as “disorders”) include, but are not limited to, dermatologic disorders, disorders of the lung, disorders of the ear, ocular disorders, disorders of the gastrointestinal tract, and disorders of the urinary tract.

The dermatologic disorders to be treated or prevented include, for example, skin disorders such as atopic dermatitis; skin photodamage; extrinsic skin aging; skin irritation; chronic, burn and ulcer wounds; acne; rosacea; psoriasis; lichen (particularly lichen planus); basal or squamous cell carcinoma (Bowen's disease); Kaposi's sarcoma; keratosis, such as actinic or seborrheic keratosis; disorders of keratinization, such as ichthyosis (particularly lamellar ichthyosis) and keratoderma. Disorders of the lung to be treated or prevented with the method include, for example, inflammation of the lung mucosa, such as asthma. The method is also useful for treating or preventing disorders of the ear and eye, such as otitis and conjunctivitis, and inflammatory disease of the gastrointestinal and urinary tract, such as colitis and interstitial cystitis.

Skin inflammation and irritation to be treated or prevented by the method can also be caused by, for example, transdermal drug delivery, irritating drug delivery enhancers or irritating drug substances that are found in pharmaceutical products as well as in skin care products.

In accordance with the method, a composition containing a protease inhibitor in a dosage sufficient to inhibit or ameliorate the disorder is administered to a human or animal in need of or desiring treatment to treat, repress or prevent the disorder. In a preferred embodiment, the composition is a pharmaceutical composition in a gel formulation for topical administration.

In addition, the method described herein is utilized in a wide variety of surgical procedures, such as the treatment of tumor excision sites on skin. For example, the composition described herein is utilized to coat, swab, brush or spray an area prior to removal of a tumor in order to prevent the spread of inflammation to surrounding tissues. Alternatively, the composition is delivered via endoscopic procedures or catheterization in order to coat tumors, or inhibit proliferation or inflammation of mucous membrane.

Methods are also provided for treating hypertrophic scars and keloids by administering the composition to a hypertrophic scar or keloid. For example, the composition is directly injected or rubbed onto a hypertrophic scar or keloid in order to prevent the progression of these lesions. This therapy is of particular value in the prophylactic treatment of conditions that are known to result in the development of hypertrophic scars and keloids (i.e., burns), and is preferably initiated after the proliferative phase has progressed, but before hypertrophic scar or keloid developed.

### Methods of Making Protease Inhibitor Containing Gel Formulations

A method of preparing a pharmaceutical composition containing a protease inhibitor is provided. Preferably, the pharmaceutical composition to be manufactured is a protease inhibitor gel formulation. The gel formulation is prepared according to a procedure that ensures suitable pH conditions within the gel, optimum protease inhibitor solubility, gel consistency, and sterility in the resulting product. The gel formulation is preferably intended for topical administration, and dosage forms are provided that are specially formulated for the therapeutic use of a protease inhibitor as a topical formulation. The dosage forms selected for topical applications should ideally release large amounts of the protease inhibitor, be sterile and non-toxic for the wound.

A preferred method of making a pharmaceutical protease inhibitor gel formulation is as follows. A powdered gelling agent is combined with an aqueous solution, such as a physiological buffer until hydrated to form a gel. The pH of the gel is adjusted to a neutral pH, preferably between approximately pH 5.5 to 9. The gel is then sterilized, preferably by irradiation. The protease inhibitor, preferably contained in a vehicle control buffer, is then mixed with the gel solution to produce the protease inhibitor gel formulation. The pH of the protease inhibitor gel is then adjusted, if necessary to a pH between approximately 5.5 and 9, preferably from about pH 6.5 to pH 8, more preferably from about pH 7 to pH 7.5, most preferably pH 7.5. A preservative is optionally added before, during or after formulation of the gel. The gel formulation is then stored under conditions that maintain sterility and activity, such as in a sealed vessel in a refrigerator or cold room.

For example, hydroxyethyl cellulose at a concentration of about 1.5% w/w is combined with 20 mM sodium phosphate at a pH of about 7.4 and stirred until fully hydrated. A recombinant alpha 1-antitrypsin (rAAT) plus a vehicle control buffer is added to the gel solution while mixing. The rAAT is normally supplied as a 5% solution in phosphate, potassium chloride, or sodium chloride, N-acetylcysteine, sodium citrate buffers at a concentration of 20 mM:200 mM:5 mM:5 mM at a pH of approximately 7.5. If appropriate, the composition additionally contains a preservative including, for example, quaternium at a concentration of about 0.02% w/w, and a pH of about 7.5. The gel formulation is then stored in airtight tubes at 4° C. until use.

In another embodiment the gel is lyophilized. A preferred gel formulation with desirable stability and activity is lyophilized and dissolved in water prior to use. Alternatively, stable gel formulations are lyophilized with reduced salt concentrations and reconstituted in sterile saline prior to use. The shelf life of the protease inhibitor gel formulation prepared in accordance with the methods described herein is preferably at least one year.

### Kits

A pharmaceutical pack or kit is also provided herein. The pack or kit includes one or more containers filled with one or more of the ingredients of the compositions described herein. Optionally associated with the container can be a notice in the form prescribed by a governmental agency regulating the manufacture, use or sale of pharmaceuticals or biological products, which notice reflects approval by the agency of manufacture, use or sale for human or veterinary administration.

### EXAMPLES

This invention is further illustrated by the following examples, including the Tables, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention or the scope of the appended claims.

The following tables demonstrate examples of antitrypsin gel formulations and corresponding activity and stability data.

TABLE 1
Stability Summary (Series 1)
Purpose: To evaluate alpha 1-antitrypsin in different aqueous gels.
Product: Alpha 1-antitrypsin Gel, 0.5%
Package Material: Clear, glass, 4 ml short vials

| | | % w/w | | | | |
|---|---|---|---|---|---|---|
| Components | | AA-6A | AA-7B | AA-14A | AA-19A | *AA-20A |
| Antitrypsin (51.65 mg/ml) | | 10.0 | 10.0 | 10.0 | 10.0 | 10.0 |
| Propylene Glycol | | — | — | — | — | 10.0 |
| Glycerin | | — | — | 20.0 | — | — |
| Benzyl Alcohol | | 1.0 | 1.0 | — | — | — |
| Methylparaben | | — | — | 0.2 | 0.2 | 0.2 |
| Propylparaben | | — | — | 0.04 | 0.04 | 0.04 |
| Hydroxyethyl cellulose (HHX) | | 2.0 | 2.0 | 2.0 | — | — |
| Carbopol 980 | | — | — | — | 0.5 | 0.5 |
| 400 mM Phosphate/Citrate Buffer | | — | 25.0 | 25.0 | — | — |
| NaOH, 10% | | — | — | — | pH to 7.5 | pH to 7.5 |
| Purified Water | | 97.0 | 62.0 | 42.76 | Qsad | Qsad |
| | | Description | | | | |
| 2 weeks | | clear | Hazy | clear | clear | clear |
| pH (1:9) | | 7.45 | 7.71 | 7.68 | 8.06 | 7.76 |
| 1 month | | N/t | n/t | n/t | clear | clear |
| 3 months | | N/t | n/t | n/t | TBD | TBD |
| | | RAAT Activity (% Label Claim) | | | | |
| T = 0 | | 97.2% | | | | |
| 2 weeks | 5° C. | N/a | n/a | 46.7 | 97.7 | 105.6 |
| | 25° C. | N/a | n/a | n/a | 99.3 | 96.2 |
| | 40° C. | N/a | n/a | n/a | n/a | n/a |
| | −20° C. | N/a | n/a | 67.1 | 98.3 | 93.2 |
| 1 month | 5° C. | N/t | N/t | n/t | 105.5 | 103.4 |
| | 25° C. | N/t | N/t | n/t | 102.3 | 88.3 |
| 2 months | 25° C. | N/t | N/t | n/t | n/t | 74.0 |
| 3 months | 5° C. | N/t | N/t | n/t | 107.5 | 88.5 |
| | 25° C. | N/t | N/t | n/t | 92.3 | 68.0 |
| | 25° C. | N/t | N/t | n/t | 92.3 | 68.0 |

Contains only 90% of targeted concentration, % w/w = 9.0%.
n/a No Activity
n/t Not Tested
TBD To Be Determined

TABLE 2
Formulation Summary of Protocol 704 (Series II)
Purpose: To evaluate time temperature stability of alpha 1-antitrypsin compounded into aqueous base formulations.
Stability: 5° C., 25° C., 30° C., Freeze/Thaw
Pull Dates: 2 weeks, 1, 3 and 6 months***
Package Material: Clear, glass, 2 ml short vials

| | | % w/w | | | | | | | | | | | | |
|---|---|---|---|---|---|---|---|---|---|---|---|---|---|---|
| | | AA | AA | AA | AA | AA | AA | AA | AA | AA | AA | AA | AA | AA |
| Components | | 42B | 43A | 44B | 45B | 46B | 47B | 48A | 49A | 50A | 51A | 52A | 53A | 57A |
| Antitrypsin (51.65 mg/ml) | | 10.0 | 10.0 | 10.0 | 10.0 | 10.0 | 10.0 | 10.0 | 10.0 | 10.0 | 60.0 | 10.0 | 10.0 | 10.0 |
| Glycerin | | | | | | 10.0 | | | | | | | | |
| Benzyl Alcohol | | | | | | | | 1.0 | | | | | | |
| Methylparaben | | 0.2 | 0.2 | 0.2 | 0.2 | 0.2 | 0.2 | | | 0.2 | 0.2 | 0.2 | 0.2 | 0.2 |
| Propylparaben | | 0.04 | 0.04 | 0.04 | 0.04 | 0.04 | 0.04 | | | 0.04 | 0.04 | 0.04 | 0.04 | 0.04 |
| Propylene Glycol | | | | | 10.0 | | 5.0 | | | | | | | |
| Ethoxydiglycol | | | | | | | 5.0 | | | | | | | |
| Bezalkonium Chloride | | | | | | | | | 0.02 | | | | | |
| Hydroxyethyl cellulose | | | | | | | | | | | | | 1.5 | |
| (HHX) | | | | | | | | | | | | | | |
| Carbopol 980 | | 0.5 | 0.5 | 0.5 | 0.5 | 0.5 | 0.5 | 0.5 | 0.5 | 1.0 | 0.5 | | | |
| Noveon AA1 | | | | | | | | | | | | 0.5 | | |
| NaOH, 10% (adjust pH to) | | 8.0 | 7.5 | 8.5 | 8.0 | 8.0 | 8.0 | 8.0 | 8.0 | 8.0 | 8.0 | 8.0 | | |
| 200 mM Phosphate/Citrate | | | | | | | | | | | | | | Qsad |
| Buffer pH 7.5 | | | | | | | | | | | | | | |
| Purified Water | | Qsad | Qsad | Qsad | Qsad | Qsad | Qsad | Qsad | Qsad | Qsad | Qsad | Qsad | Qsad | |
| | | Description | | | | | | | | | | | | |
| | | Clear | Clear | Clear | Clear | Clear | Clear | Cloudy | Clear | Cloudy | Cloudy | Clear | Clear | Clear |
| pH (1:9) | | 7.95 | 7.67 | 8.34 | 8.00 | 7.92 | 7.99 | 8.09 | 8.02 | 8.30 | 8.11 | 7.72 | 7.01 | *7.42 |
| | | rAAT Activity (% Label Claim) | | | | | | | | | | | | |
| 2 weeks | 5° C. | 1.4 | n/t | 19.8 | n/t | 6.5 | n/t | n/t | n/t | n/t | 18.5 | n/t | n/t | 6.9 |
| | 25° C. | 3.6 | n/t | 30.5 | n/t | 3.5 | n/t | n/t | n/t | n/t | 8.6 | n/t | n/t | 2.7 |
| | 30° C. | 2.2 | n/t | 6.2 | n/t | −2.1 | n/t | n/t | n/t | n/t | −0.3 | n/t | n/t | 0.0 |
| | F/Thaw | 3.1 | n/t | n/t | n/t | 3.4 | n/t | n/t | n/t | n/t | 2.2 | n/t | n/t | 1.2 |

*pH taken neat (solution)
n/t Not Tested.
***Samples were no longer submitted after 2 week results.

TABLE 3
Formulation Summary of Protocol 710 (Series III)
Purpose: To evaluate time temperature stability of Alpha 1-antitrypsin compounded into aqueous base formulations.
Stability: 5° C., 25° C., 30° C., Freeze/Thaw
Pull Dates: 2 weeks, 1, 3 and 6 monthsPackage Material: Clear, glass, 2 ml short vials

| | | % w/w | | | | | | | | | |
|---|---|---|---|---|---|---|---|---|---|---|---|
| Components | | AA-62A | AA-63A | AA-64A | AA-65A | AA-66A | AA-67A | AA-68A | AA-69A | AA-71A | AA-72A |
| Antitrypsin (51.65 mg/ml) | | 10.0 | 10.0 | 10.0 | 10.0 | 10.0 | 10.0 | 10.0 | 10.0 | 10.0 | 10.0 |
| Propylene Glycol | | — | 10.0 | — | — | 10.0 | — | — | — | — | — |
| Glycerin | | — | — | 10.0 | 20.0 | 10.0 | — | — | — | — | — |
| Benzyl Alcohol | | — | — | — | — | — | 1.0 | — | 1.0 | — | — |
| Methylparaben | | 0.2 | 0.2 | 0.2 | 0.2 | 0.2 | — | 0.2 | — | — | 0.2 |
| Propylparaben | | 0.04 | 0.04 | 0.04 | 0.04 | 0.04 | — | 0.04 | — | — | 0.04 |
| Dowicil 200 | | — | — | — | — | — | — | — | — | 0.02 | — |
| Hydroxyethyl cellulose | | — | — | — | — | — | — | 1.5 | 1.5 | 1.5 | 1.5 |
| (HHX) | | | | | | | | | | | |
| Carbopol 980 | | 0.5 | 0.5 | 0.5 | 0.5 | 0.5 | 0.5 | — | — | — | — |
| NaOH, 10% (adjust pH to) | | 8.5 | 8.5 | 8.5 | 8.5 | 8.5 | 8.5 | — | — | — | — |
| 200 mM Phosphate/Citrate | | — | — | — | — | — | — | — | — | — | Qsad |
| Buffer pH 7.5 | | | | | | | | | | | |
| 200 mM Phosphate/Citrate | | — | — | — | — | — | — | Qsad | Qsad | Qsad | — |
| Buffer pH 8.5 | | | | | | | | | | | |
| Purified Water | | Qsad | Qsad | Qsad | Qsad | Qsad | Qsad | — | — | — | — |
| | | Description | | | | | | | | | |
| | | Clear | Clear | Clear | Clear | Clear | Clear | Clear | Cloudy | Clear | Cloudy |
| PH (1:9) | | 8.43 | 8.30 | 8.41 | 8.33 | 8.41 | 8.58 | 8.03 | 8.32 | 8.41 | 7.59 |
| | | rAAT Activity (% label claim) | | | | | | | | | |
| T = 0 | | 59.8 | 112.1 | 101.8 | 106.7 | 109.5 | 77.2 | 71.6 | 11.5 | 101.7 | 74.4 |
| 2 weeks | 5° C. | 6.5 | 33.3 | 63.1 | 103.3 | 93.9 | n/t | 5.1 | n/t | 94.8 | n/t |
| | 30° C. | −1.0 | 0.5 | 19.9 | 70.6 | −0.1 | n/t | −3.8 | n/t | 43.7 | n/t |
| | F/Thaw | −0.8 | 3.6 | 41.5 | 65.6 | 38 | n/t | 1.1 | n/t | 81.1 | n/t |
| 1month | 5° C. | n/t | n/t | n/t | 86.0 | 73.6 | n/t | n/t | n/t | 93.7 | n/t |
| | 30° C. | n/t | n/t | n/t | 36.6 | n/t | n/t | n/t | n/t | n/t | n/t |
| 3 months | 5° C. | n/t | n/t | n/t | 77.9 | 50.2 | n/t | n/t | n/t | 82.3 | n/t |

n/t Not Tested.

TABLE 4
Antitrypsin Formulation Summary of Protocol 713 (Series IV)

| | | % w/w | | | | | | | | | |
|---|---|---|---|---|---|---|---|---|---|---|---|
| Components | | AA-78A | AA-79A | AA-80A | AA-81A | AA-82A | AA-83A | AA-84A | AA-85A | AA-86A | *AA-87A |
| Antitrypsin (51.65 mg/ml) | | 10.0 | 10.0 | 10.0 | 10.0 | 10.0 | 10.0 | 10.0 | 10.0 | 10.0 | 10.0 |
| Propylene Glycool | | | 10.0 | 10.0 | 10.0 | 10.0 | 10.0 | | | | |
| Glycerin | | | | | | | | 10.0 | 40.0 | 60.0 | 10.0 |
| Methylparaben | | | | 0.2 | 0.2 | 0.2 | 0.2 | | | | |
| Propylparaben | | | | 0.04 | 0.04 | 0.04 | 0.04 | | | | |
| Quaternium 15 | | 0.02 | 0.02 | | | | | 0.02 | 0.02 | | 0.02 |
| (Dowicil 200) | | | | | | | | | | | |
| Trolamine | | | | | | | | qsad | qsad | qsad | qsad |
| | | | | | | | | pH 8.3 | pH 8.3 | pH 8.3 | pH 8.3 |
| Hydroxyethyl cellulose | | 1.5 | 1.5 | 1.5 | 1.5 | 1.5 | 1.5 | | | | |
| (HHX) | | | | | | | | | | | |
| Carbopol 980 | | | | | | | | 0.5 | 0.5 | 0.5 | 0.5 |
| 200 mM Phosphate Buffer | | 88.48 | 78.48 | | | | | | | | |
| Client Buffer (pH 7.0) | | | | 78.26 | | | | | | | |
| Client Buffer (pH 7.5) | | | | | 78.26 | | | | | | |
| Client Buffer (pH 8.0) | | | | | | 78.26 | | | | | |
| Tris Buffer (pH 8.0) | | | | | | | 78.26 | | | | |
| Purified Water | | | | | | | | qsad 100 | qsad 100 | qsad 100 | qsad 100 |
| | | Description | | | | | | | | | |
| | | Clear | Clear | S. Hazy | Clear | S. Hazy | S. Hazy | Clear | Clear | Bubbles | Clear |
| pH (1:9) | | 8.26 | 8.23 | 7.34 | 7.44 | 7.71 | 7.82 | 7.94 | 8.02 | 8.15 | 8.20 |
| | | rAAT Activity (% label claim) | | | | | | | | | |
| T = 0 | | 111.0 | 98.2 | 90.3 | 76.6 | 105.4 | 89.6 | 122.9 | 99.0 | 137.8 | 110.5 |
| 2 Weeks | 5° C. | 78.8 | 61.5 | 70.0 | 15.9 | 72.3 | 12.4 | 79.3 | −0.3 | 101.4 | 105.0 |
| | 25° C. | 58.7 | 43.0 | n/t | n/t | n/t | n/t | 64.8 | 65.9 | 39.3 | 83.0 |
| | 30° C. | 16.1 | 9.8 | n/t | n/t | n/t | n/t | 49.2 | 47.5 | 60.8 | 56.3 |
| | F/Thaw | 90.2 | 64.9 | n/t | n/t | n/t | n/t | 67.2 | 72.8 | 90.3 | 87.5 |
| 1 month | 5° C. | n/t | n/t | n/t | n/t | n/t | n/t | 99.4 | 82.4 | 96.6 | 98.3 |
| | 25° C. | n/t | n/t | n/t | n/t | n/t | n/t | 49.6 | 41.4 | N/t | 58.5 |

n/t Not tested
***3 Month samples not submitted.

TABLE 5
Antitrypsin Formulation Summary of Protocol (Series V)

| | | AA- | AA- | AA- | AA- | AA- | AA- | AA- | AA- | AA- | AA- |
|---|---|---|---|---|---|---|---|---|---|---|---|
| Components | | 96A | 97A | 99A | 100A | 101A | 103A | 104A | 105A | 106A | 108A |
| Antitrypsin (51.65 mg/ml) | | 10.0 | 10.0 | 10.0 | 10.0 | 10.0 | 10.0 | 10.0 | 10.0 | 10.0 | 10.0 |
| Propylene Glycol | | | | | | | | | | | |
| Glycerin | | | | 10.0 | 10.0 | | | | | | |
| Tween 20 | | | | | | | | | | 0.5 | |
| Methylparaben | | | | | | | 0.18 | 0.18 | | | 0.18 |
| Propylparaben | | | | | | | 0.036 | 0.036 | | | 0.036 |
| Quaternium 15 (Dowicil 200) | | 0.02 | 0.02 | 0.02 | 0.02 | 0.02 | | | 0.02 | 0.02 | |
| Trolamine | | | | 1.66 | 1.66 | | | | | | |
| Hydroxyethyl cellulose (HHX) | | 1.5 | 1.5 | | | 1.5 | | | | | |
| Carbopol 980 | | | | 0.5 | 0.5 | | 0.45 | 0.45 | | | 0.45 |
| 10% NaOH | | | | | | | 2.0 | 2.0 | | | 2.0 |
| Buffer #1, pH 8.2, 50 mM KCl | | | 88.48 | | | | | | | | |
| Buffer #2, pH 8.2 | | | | 88.48 | | | | | | | |
| Buffer #3, pH 7.4, 50 mM KCl | | | | | | | | | 89.98 | | |
| Buffer #4, pH 7.4 | | | | | | | | | | 89.98 | |
| Buffer #5, pH 8.2, 20 mM | | | | | | 88.48 | | | | | |
| NaPO4 | | | | | | | | | | | |
| Purified Water | | | | 77.88 | 77.88 | | 87.334 | 87.334 | | | 86.834 |
| | | Description | | | | | | | | | |
| | | clear | clear | clear | clear | clear | clear | clear | clear | clear | cloudy |
| pH (1:9) | | 8.00 | 8.13 | 8.02 | 8.06 | 8.07 | 7.81 | 7.84 | n/t | n/t | 7.82 |
| | | rAAT Activity (% label claim) | | | | | | | | | |
| T = 0 | | 101.4 | 99.7 | 106.6 | 135.8 | 95.7 | n/t | n/t | 107.5 | 106.2 | n/t |
| 2 Weeks | 5° C. | 91.4 | 102.8 | 97.2 | 119.0 | 105.8 | n/t | n/t | 104.1 | 104.8 | N/t |
| | 25° C. | 77.1 | 136.0 | 79.7 | 102.8 | 92.5 | n/t | n/t | 101.0 | 97.7 | N/t |
| 1 month | 5° C. | 83.4 | 76.4 | 85.3 | 97.3 | 89.9 | n/t | n/t | 92.5 | 92.0 | N/t |
| | 25° C. | 37.8 | 27.7 | 53.5 | 65.6 | 89.3 | n/t | n/t | 81.6 | 72.1 | N/t |
| 3 months | 5° C. | n/t | n/t | n/t | n/t | 74.3 | n/t | n/t | 85.7 | 83.5 | N/t |
| | 25° C. | n/t | n/t | n/t | n/t | n/t | n/t | n/t | n/t | 31.0 | N/t |

n/t Not tested

TABLE 6
Antitrypsin Formulation Summary of Protocol (Series VI)

| | | % w/w | | | | | | | | | | | | | |
|---|---|---|---|---|---|---|---|---|---|---|---|---|---|---|---|
| Components | | 19A | 19A2 | 65A | 66A | 66A2 | 84A | 84A2 | 85A | 86A | 71A | 97A | 101A | (A) | (B) |
| Antitrypsin (51.65 mg/ml) | | 10.0 | 10.0 | 10.0 | 10.0 | 10.0 | 10.0 | 10.0 | 10.0 | 10.0 | 10.0 | 10.0 | 10.0 | 10.0 | 10.0 |
| Propylene Glycol | | | | | 10.0 | 10.0 | | | | | | | | | |
| Glycerin | | | | 20.0 | 10.0 | 10.0 | 10.0 | 10.0 | 40.0 | 60.0 | | | | | |
| Methylparaben | | 0.2 | 0.2 | 0.2 | 0.2 | 0.2 | | | | | | | | | |
| Propylparaben | | 0.04 | 0.04 | 0.04 | 0.04 | 0.04 | | | | | | | | | |
| Quaternium 15 (Dowicil | | | | | | | 0.02 | 0.02 | 0.02 | 0.02 | 0.02 | 0.02 | 0.02 | 0.02 | 0.02 |
| 200) | | | | | | | | | | | | | | | |
| Trolamine | | | | | | | 1.65 | 1.65 | 1.65 | 1.65 | | | | | |
| Hydroxyethyl cellulose | | | | | | | | | | | 1.5 | 1.5 | 1.5 | 1.5 | 1.5 |
| (HHX) | | | | | | | | | | | | | | | |
| Carbopol 980 | | 0.5 | 0.5 | 0.5 | 0.5 | 0.5 | 0.5 | 0.5 | 0.5 | 0.5 | | | | | |
| 10% NaOH | | 2.0 | 2.0 | 2.9 | 2.9 | 2.9 | | | | | | | | | |
| N-Acetylcysteine | | | 0.08 | | | 0.08 | | 0.08 | | | | | | | |
| 5 mM Sodium Citrate, 20 mM | | | | | | | | | | | | 88.48 | | | |
| Na₂HPO4 and 5 mM N- | | | | | | | | | | | | | | | |
| Acetylcysteine, pH 8.2 | | | | | | | | | | | | | | | |
| 5 mM Sodium Citrate, 20 mM | | | | | | | | | | | | | | 88.48 | |
| Na₂HPO4 and 5 mM | | | | | | | | | | | | | | | |
| N-Acetylcysteine, pH 7.4 | | | | | | | | | | | | | | | |
| 20 mM NaPO4 - pH 8.2 | | | | | | | | | | | | | 88.48 | | |
| 20 mM NaPO4 - pH 7.4 | | | | | | | | | | | | | | | 88.48 |
| Buffer #7, pH 8.5, | | | | | | | | | | | 88.48 | | | | |
| 200 mM Sodium/Citrate | | | | | | | | | | | | | | | |
| Purified Water | | 87.26 | 87.18 | 66.36 | 66.36 | 66.28 | 77.83 | 77.75 | 47.83 | 27.83 | | | | | |
| | | Description | | | | | | | | | | | | | |
| pH (1:9) | | 7.86 | 7.95 | 8.37 | 8.25 | 8.15 | 8.05 | 7.94 | 8.06 | 8.04 | 8.48 | 8.18 | 8.05 | 7.52 | 7.60 |
| | | rAAT Activity (% label claim) | | | | | | | | | | | | | |
| T = 0 | | 2.3 | 2.9 | 92.3 | 84.2 | 86.0 | 96.2 | 102.7 | 90.8 | 101.5 | 93.2 | 92.1 | 97.3 | 96.8 | 96.8 |
| 2 weeks | 5° C. | 1.7 | 2.1 | 91.6 | 71.9 | 59.3 | 89.9 | 97.7 | 88.8 | 95.5 | 97.2 | 85.8 | 94.3 | 95.1 | 93.6 |
| | 25° C. | 3.6 | 4.1 | 89.3 | 35.3 | 26.3 | 70.8 | 82.7 | 74.6 | 81.6 | 50.7 | 80.1 | 64.8 | 91.7 | 80.1 |
| 1 month | 5° C. | n/t | n/t | 93 | 64.2 | 56.6 | 87.6 | 100.2 | 88.4 | 92.9 | 86.8 | n/t | 90.5 | 95.4 | 92.5 |
| | 25° C. | n/t | n/t | 77.9 | 17.4 | 15.1 | 58.4 | 84.7 | 63.2 | 66.9 | 24.0 | 4.5 | 40.1 | 60.9 | 50.9 |
| 2 months | 5° C. | n/t | n/t | 74.3 | 49.4 | n/t | n/t | 96.6 | 82.6 | 72.3 | 64.4 | n/t | 82.0 | 73.1 | 88.9 |
| | 25° C. | n/t | n/t | 68.6 | 8.6 | n/t | n/t | 68.4 | 49.8 | 47.6 | 18.3 | n/t | 45.4 | | 55.5 |

TABLE 7
Antitrypsin Gel Formulations Series VII

| Components | | 785-8A | 785-9A | 785-10A | 785-11A | 785-12A | 785-13A | 785-14A | 785-15A | 785-16A |
|---|---|---|---|---|---|---|---|---|---|---|
| Antitrypsin (51.65 mg/ml) | | 10.0 | 10.0 | 10.0 | 10.0 | 10.0 | 10.0 | 10.0 | 10.0 | 10.0 |
| Methylparaben | | 0.2 | 0.2 | | | | | | | |
| Propylparaben | | 0.04 | 0.04 | | | | | | | |
| Quaternium 15 (Dowicil 200) | | | | 0.02 | 0.02 | 0.02 | 0.02 | 0.02 | 0.02 | 0.02 |
| Propylene Glycol | | | 10.0 | | | | | | | |
| Glycerin | | 20.0 | 10.0 | 10.0 | 40.0 | 60.0 | | | | |
| Carbopol 980 | | 0.5 | 0.5 | 0.5 | 0.5 | 0.5 | | | | |
| Hydroxyethyl cellulose HHX | | | | | | | 1.5 | 1.5 | 1.5 | 1.5 |
| NaOH, 10% | | 2.9 | 2.9 | | | | | | | |
| Trolamine | | | | 1.65 | 1.65 | 1.65 | | | | |
| N-Acetylcysteine | | | | 0.08 | | | | | | |
| 200 mM Phosphate/Citrate Buffer | | | | | | | 88.48 | | | |
| (pH 8.5) | | | | | | | | | | |
| 20 mM Phosphate Buffer (pH 8.2) | | | | | | | | 88.48 | | |
| 20 mM Phosphate Buffer (pH 7.4) | | | | | | | | | | 88.48 |
| *Buffer pH 7.4 | | | | | | | | | 88.48 | |
| Purified Water | | 66.36 | 66.36 | 77.75 | 47.83 | 27.83 | | | | |
| | | Description | | | | | | | | |
| | | Clear | Clear | Clear | Clear | Tiny | Clear | Clear | Clear | Clear |
| | | | | | | Bubbles | | | | |
| | | | | | | Hazy | | | | |
| | | | | | | appear. | | | | |
| pH (1:9) | | 8.33 | 8.34 | 8.10 | 7.97 | 8.01 | 8.43 | 7.95 | 7.87 | 7.45 |
| | | rAAT Activity (% Label Claim) | | | | | | | | |
| T = 0 | | 124.5 | 75.6 | 97.3 | 88.5 | 97.1 | 97.5 | 99.7 | 97.2 | 98.5 |
| 2 weeks | 5° C. | 110.5 | 39.1 | 82.5 | 82.5 | 63.5 | 94.5 | 113.7 | 95.3 | 104.2 |
| | 25° C. | 97.7 | 12.9 | 51.6 | 63.9 | 48.8 | 45.4 | 90.0 | 88.2 | 88.4 |
| 1 month | 5° C. | 102.8 | 39.5 | 83.5 | 84.6 | 82.3 | 91.6 | 91.7 | 95.2 | 95.6 |
| | 25° C. | 94.4 | 7.6 | 35.0 | 55.1 | 38.3 | 26.7 | 91.7 | 82.7 | 92.4 |

*5 mM Sodium Citrate, 20 mM Na₂HPO₄ and 5 mM N-Acetylcysteine

TABLE 8
Stability Data For rAAT Formulations

| | | rAAT Activity (% label claim)* | | | | | | | | | |
|---|---|---|---|---|---|---|---|---|---|---|---|
| | | Time (months) | | | | | | | | | |
| % rAAT/Batch | Temperature | | 0 | 0.5 | 1.0 | 2.0 | 3.0 | 4.0 | 5.0 | 6.0 | 8.0 |
| 0.25/785-66A | 5° C. | plastic | 115.4 | 112.7 | 108.6 | 105.1 | 101.6 | 103.2 | | 96.9 | |
| 0.25/785-66A | (real time) | glaminate | | 114.4 | 106.9 | 103.0 | 103.6 | 102.4 | | 95.2 | |
| 0.25/785-66A | 25° C. | plastic | | 106.9 | 75.5 | X | X | | | | |
| 0.25/785-66A | (accelerated) | glaminate | | 104.6 | 77.0 | X | X | | | | |
| 0.5/773-139A | 5° C. | | 98.7 | 89.1 | 85.4 | 88.9 | 85.1 | | | 88.9 | 84.2 |
| | (real time) | | | | | | | | | | |
| 0.5/773-139A | 25° C. | | | 79.6 | 54.2 | 50.0 | 37.4 | | | 27.7 | |
| | (accelerated) | | | | | | | | | | |
| 2.0/785-70A | 5° C. | plastic | 99.5 | X | 92.7 | 90.5 | 92.1 | X | 90.2 | | |
| 2.0/785-70A | (real time) | glaminate | | X | 93.2 | 91.0 | 90.1 | X | 91.0 | | |
| 2.0/785-70A | 25° C. | plastic | | X | 86.4 | | | | | | |
| 2.0/785-70A | (accelerated) | glaminate | | X | 88.8 | | | | | | |

Note:
no stability data on T = 0.5 for batch 785-70A

### Example 1

### Determination of the Specific Activity of Recombinant Alpha 1-Antitrypsin

The purpose of this study was the release and stability testing of recombinant alpha 1-antitrypsin (rAAT) in the pharmaceutical composition. This method may also be used as a qualitative identity test for rAAT as an elastase inhibitor in gels, 0.1% to 3.0%, by measuring the inhibitory effect of rAAT on porcine pancreatic elastase (PPE). The assay is performed in a microtiter plate.

REAGENTS: Tris-HCI: FW 157.6 g/mole, Electerophoresis grade, Fisher Cat# BP153-500. Tris-base: FW 121.1 g/mole, biotechnology performance certified, Sigma Cat# T 6066. Water, HPLC grade. sodium chloride: FW 58.44 g/mole, certified A.C.S, Bovine Serum Albumin (BSA): Fraction V. Protease-free, Golden West, Cat# BA 1060. Porcine Pancreatic Elastase, Grade II Lyophilized, Roche Diagnostic Corp. Cat# 100907. N-Suc-Ala-Ala-Ala-pNA, -Bachem, Cat# I-1385. rAAT Reference Standard, Arriva Pharmaceuticals.

SOLUTIONS: 100 mM Tris-Base: Dissolve 12.1 g Tris-Base to 1.0 L volume with HPLC grade water. Mix well and filter this solution through 0.22 μm cellulose acetate filter (disposable filter system) under vacuum. Storage: for up to 6 months. 100 mM Tris-HCl: Dissolve 15.8 g Tris-HCl to 1.0 L volume with HPLC grade water. Mix well and filter this solution through 0.22 μm cellulose acetate filter under vacuum. Storage: for up to six months. Note: The above two solutions are used for PH adjustment of other solutions.

PPE Buffer, 100 mM Tris-HCl solution, pH 8.0: Combine 1.37 g of Tris-HCI, 0.236 g of Tris-Base and approximately 60 ml of purified water. Adjust pH to 8.0 (±0.05) with 100 mM Tris-Base or 300 mM Tris-HCI. Dilute to 100 ml volume with HPLC grade water. Mix well and filter this solution through 0.22 μm cellulose acetate filter under vacuum. Storage: for up to six months.

Assay Buffer: 50 mM Tris-HCI, 150 mM Tris-Base, pH 8.0, 0.5 M NaCl, 0.01% BSA (Bovine Serum Albumin):

Step 1: Combine 6.35 g of Tris-HCI, 3.18 g of Tris-Base and approx. 600 ml of purified water. Mix well and adjust pH to 8.0 (±0.05) with 100 MM Tris-Base or 100 mM Tris-HCI.

Step 2: Add 29.22 g of NaCl first, then add 0.1 g BSA to the pH 8.0 buffer, Mix until dissolved. Dilute to 1.0 L volume with HPLC grade water. Filter this solution through 0.22 μm cellulose acetate filter under vacuum. Storage: 2-8° C. for up to two months.

PPE Stock Solution, 100 μg/ml: Dissolve 25 mg of Porcine Pancreatic Elastase to 250 ml volume with the PPE Buffer, and mix well. Smaller proportional volumes can be prepared. Decant into 4 ml volumes. Store at −80° C. for up to 6 months. Do not freeze-thaw this solution more than once. Elastase activity (U/mg protein) is in the C of A.

PPE Cocktail, 3 μg/ml: Dilute 3.0 mL of the PPE Stock Solution to 300 ml volume with the Assay Buffer and mix well. Decant into 4 mL volumes. Store at −80° C. for up to six months. Do not freeze-thaw this solution more than once.

Substrate Solution, N-Suc-Ala-Ala-Ala-pNA 2.7 mg/ml: Dissolve. 270 mg of N-Suc-Ala-Ala-Ala-pNA to 100 ml volume with the Assay Buffer. Decant into 4 ml volumes. Store at −80° C. for up to six months. Do not freeze-thaw this solution more than once. Substrate solution is stable for 12 hours at 25° C.

TABLE 9
Method of diluting rAAT standard solution in assay buffer
Prepare 1.722 μg/ml rAAT from 51.65 mg/ml rAAT concentrate

| Start | | Dil. vol. (ml) | | Dilution |
|---|---|---|---|---|
| mg/ml | Ml assay buffer | Assay | Net | factor per |
| [C of A] | (mg/ml rAAT gel) | buffer | con. mg/ml | ml Rx mix |
| 51.65 | 5 (51.65) | 10 | 25.825 | |
| | 4 (25.825) | 10 | 10.330 | |
| | 1 (10.330) | 100 | 0.103 | |
| | 10 (0.103) | 100 | 0.0103 | |
| | 10 (0.01033) | 60 | 1.722 μg/ml | 599880 |

To prepare 1.722 μg/ml rAAT from 51.65 mg/ml rAAT reference standard: Dilute 5.0 ml of 51.65 mg/ml to 10.0 ml volume, net 25.825 mg/ml. Dilute 4.0 ml of 25.825 mg/ml to 10.0 ml volume, net 10.330 mg/ml. Dilute 1.0 ml of 10.330 mg/ml to 100.0 ml volume, net 0.103 mg/ml. Dilute 10.0 ml of 0.103 mg/ml to 100.0 ml volume, net 0.0103 mg/ml. Dilute 10.0 ml of 0.0103 mg/ml to 60.0 ml volume, net 1.722 μg/ml. Two separate dilution pools are prepared, one designated Cal and the other as Chk. Decant into 4 ml volumes. Store at −80° C. for up to six months. Do not freeze-thaw this solution more than once.

TABLE 10
Summary table for dilution of rAAT gels in assay buffer to a target
concentration of 1.7 μg/ml

| Label claim | | | | | | |
|---|---|---|---|---|---|---|
| mg rAAT | 0.5 g + 50 mL | 1 mL + 9 mL | Aliquot of | | Net | Dil. Factor |
| Per | assay buffer | assay buffer | last dilution | Dil. Vol. | Conc. | per ml Rx |
| 1000 mg gel | μg/ml | μg/ml | Ml | ml | μg/ml | mixture |
| 30 (3.0%) | 300 | 30 | 1.0 | 20.0 | 1.47 | 200000 |
| 25 (2.5%) | 250 | 25 | 1.0 | 15.0 | 1.67 | 150000 |
| 10 (1.0%) | 100 | 10 | 1.0 | 6.0 | 1.667 | 59880 |
| 5 (0.5%) | 50 | 5 | 2.0 | 6.0 | 1.667 | 59880 |
| 1 (0.1%) | 10 | | 2.0 | 12.0 | 1.67 | 11976 |

To prepare rAAT for EIA assay from 0.5% rAAT hydrophilic gel samples, dilute in Assay Buffer approximately 1.7 μg/ml rAAT. Solubilize 500±50 mg of 0.5% rAAT gel in 50.0 ml, net approximately 50 μg/ml. Dilute 1.0 ml of 50 μg/ml to 10.0 ml volume, net 5 μg/ml. Dilute 2.0 ml of 5 μg/ml to 6.0 ml volume, net 1.7 μg/ml. The overall dilution factor per ml plate well reaction mixture is 59880. The EIA may be run immediately. The final sample dilutions may be stored 5° C. and run within 24 hours.

Plate Assay Procedure: Set the internal temperature of the Microplate Reader to 30° C. and set the wavelength to 405 nm. Set up the plate template for the VERSAmax™ plate reader in SOFTmax® PRO. Allow 30 minutes for thermal equilibration. All solutions must be equilibrated at room temperature.

Calculations: Raw absorbance values (11 per well) are collected from each plate well at 1.0 min. intervals during the 10 min. kinetic assay. The SOFTmax® PRO (V3.1.2) software accumulates the absorbance values and calculates a slope (rate of color development as the substrate is hydrolyzed by PPE. To convert the data to a familiar, reviewable form, it is necessary to export the raw absorbance values into Excel®, then complete all calculations using formulas embedded in the Excel® tables. Export the raw absorbance values from SOFTmax® PRO (v3.1.2) to Excel®. Calculate the rate of absorbance change (slope) in each single well for the interval 0.0 to 10.0 min. Calculate the mean rate for the triplicate runs. Calculate the % CV. Determine Cal/Chk % agreement. The rate of color generation for the PPE enzyme control must fall within 55 to 80 mAU/min. The inhibition of enzymatic activity for samples and standards should fall between 30% and 70%, which is the heart of the linear range of the assay. The interwell CV values for the PPE control, the rAAT standards and the rAAT samples must be less than 7.5%. The mean of the substrate control replicates must represent less than 1.0% of the mean of the PPE enzyme control.

Table 11 shows the release specifications for the gel formulation of alpha 1-antitrypsin.

TABLE 11
Release specifications for alpha 1-antitrypsin gels (0.25%-2% w/w)

| Test | Specification | Method |
|---|---|---|
| Appearance | Translucent, colorless | Visual |
| | gel | |
| Identification | Conforms to standard | Elastase Inhibition Assay |
| pH | 7.5 ± 0.5 | pH |
| Viscosity | Record | TBD |
| Assay, rAAT (activity) | 80-120% Label Claim | Elastase Inhibition Assay |
| Assay, rAAT | 90-110% Label Claim | UV Absorbance Assay |
| (total protein) | | |
| Antimicrobial | Record | USP 24 <51> |
| Effectiveness Test | | |
| Endotoxin LAL | Record | USP 24 <85> |

NMT = not more than

### Example 2

### In Vitro Percutaneous Absorption of Antitrypsin in Human Cadaver Skin

The purpose of this study was to evaluate the in vitro percutaneous absorption of nine topical alpha 1-antitrypsin (0.5%) gel formulations in intact human cadaver skin. The test formulations are applied as a single dose. Transdermal absorption was measured at 1, 6 and 24 hours, epidermal, dermal and stratum corneum (S.C.) recoveries are measured at 24 hours. The experimental design is set forth below in Table 12.

TABLE 12
Experimental design

| | | | | Drug | | Epidermal Dermal |
|---|---|---|---|---|---|---|
| | % | | | Application | Reservoir | S.C. Recovery |
| Drug | Antitrypsin | N | Vehicle | (Hr) | Penetration (Hr) | (HR) |
| 785-8A | 0.5 | 6 | Gel | 0 | 1, 24 | 24 |
| 785-9A | 0.5 | 6 | Gel | 0 | 1, 6, 24 | 24 |
| 785-10A | 0.5 | 6 | Gel | 0 | 1, 6, 24 | 24 |
| 785-11A | 0.5 | 6 | Gel | 0 | 1, 6, 24 | 24 |
| 785-12A | 0.5 | 6 | Gel | 0 | 1, 6, 24 | 24 |
| 785-13A | 0.5 | 6 | Gel | 0 | 1, 6, 24 | 24 |
| 785-14A | 0.5 | 6 | Gel | 0 | 1, 6, 24 | 24 |
| 785-15A | 0.5 | 6 | Gel | 0 | 1, 6, 24 | 24 |
| 785-16A | 0.5 | 6 | Gel | 0 | 1, 6, 24 | 24 |

Radiolabeled ¹²⁵I antitrypsin (100 Ci/ml)(S.A. 53 Ci/mg) was provided by Amersham PLC.

Human Skin: Human cadaver skin (# 000504) was obtained from a single donor. The skin was dermatomed to approximately 200 micron thickness. Skin was excluded if it was damaged, had irregularities (scar tissue, holes, birthmarks, etc.) or was from donors with infectious disease. Skin samples were frozen until use. Skin specimens were thawed overnight in the refrigerator in plastic sealed bags prior to the experiment.

Test formulations were spiked with ¹²⁵I alpha 1-antitrypsin prior to the experiment. Test formulations, as indicted in Table 7, are represented by batch Nos. 785-8A, 785-9A, 785-10A, 785-11A, 785-12A, 785-13A, 785-14A, 785-15A, and 785-16A. To assay the final specific activity of the spiked test formulation, approximately 10-50 mg of the formulation was weighed and dissolved in 1.0 ml of Solusol. 100 μl samples were then placed in 10 ml of Ecoscint® scintillation fluor (National Diagnostics #LS275) and counted in a Beckman Model LS 3801 liquid scintillation counter with a pre-calibrated quench curve for ¹²⁵I.

A total of 54 Franz static diffusion glass diffusion chambers (Crown Glass Cat # FDC-100) with a magnetic stirrer mounted on a 9-position Franz diffusion cell drive console with acrylic blocks, magnetic stirrers, and stainless steel manifolds (Crown #FDCD-9-LV) was used for the study. The reservoir volume (6-11 ml) of each cell is pre-calibrated. The diffusion cells were filled with isotonic phosphate buffered (pH 7.4) saline with care to avoid bubbles at the skin interface. The diffusion cells were equilibrated for 1-2 hours to a temperature of 37° C. by a circulating water pump prior to applying skin specimens.

Application of Drug to Skin: The dermatomed human cadaver skin was placed on the chamber and sealed with an O-ring. The skin surface area exposed to the test formulations was 1.77 cm². A total of 30 mg of the spiked formulation was applied to the skin surface using a Gilson Microman® positive displacement pipette and gently rubbed into the skin using the pipette tip. The dispensing tips were retained and counted. The mean DPM retained by the dispensing tips was calculated and subtracted from the theoretical DPM to determine the mean total DPM applied to each chamber.

Sample Collection: At 1, 6 and 24 hours a 1.0 ml sample was removed from the reservoir using a calibrated Gilson P1000 Pipetteman® micropipette, and the volume replaced with 1.0 ml saline solution. The samples were placed in a scintillation vial containing Ecoscint® scintillation fluor Rational. Diagnostics # LS-275) and equilibrated overnight in the dark before counting. At 24 hours the skin surface was washed three times with 1.0 ml 2% Oleth-20 (Croda, Inc; # 9004-98-2) in water, followed by 2 washes with 1.0 ml of 5% Span 80 in isopropanol. The wash solutions were collected for recovery counts. The dispensing tips used for the washing procedure were also counted and included in the “wash” compartment. After washing, the skin surface was wiped with 3 sequential cotton gauze cloths, which were saved for recovery counts in the “Gauze” compartment.

Tape Stripping (Stratum corneum): The skin specimens were removed from the chamber and placed dermis side down onto a flat surface. The stratum corneum was removed by tape-stripping the skin with cellophane tape until “glistening” (approximately 22 strips) or until epidermal separation started to occur. The first two strips that remove the excess drug adhering to the outer surface of the stratum corneum were counted separately. These counts were included in total recovery (SC Surface) but excluded from stratum corneum compartment recovery. Four groups each consisting of five consecutive tape strips were placed in a scintillation vial containing Scintilene® (Fisher # SX2-4). After tape stripping, the dermis and epidermis were separated by the microwave technique (2-5 sec.). The separated epidermis was placed in a vial containing 1 ml of Soluene 350 (Packard, Inc # 6003038), and dermis in vial containing 2 ml Soluene 350. The tissues were then digested in a 60° C. oven for 4 hours. Hionic Fluor (Packard, Inc # 6013319) was added to the digested tissues, counted in a Beckman LSC and corrected for quenching.

The amount of alpha 1-antitrypsin recovered in the reservoir, washes, gauze wipes, and skin compartments (stratum corneum, epidermis, dermis) was determined by calculating the amount of the total applied DPM recovered in the respective compartments. The total micrograms of drug recovered in each compartment was obtained by multiplying the % recovery by the total amount (micrograms) of drug applied to each chamber. The DPM data from the LSC tapes, measurements for cell volume, and quantity of drug applied were entered into a standardized Quattro Pro spreadsheet. The data entry and computer calculations were verified to be accurate. The calculations, tables and graphs generated by the spreadsheet were spot checked to insure accuracy. In the report the data were calculated after discarding outlier values for each compartment. A value was considered to be an “outlier” if the questionable value differed from the re-calculated average by more than four times the average deviation of the remaining values (Skoog & West, Analytical Chemistry).

### RESULTS

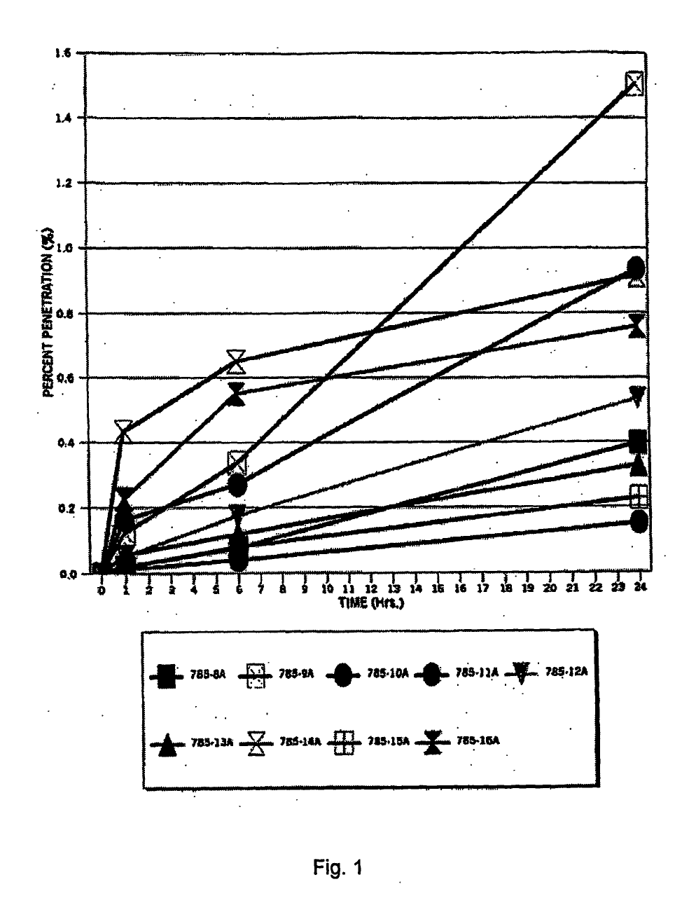

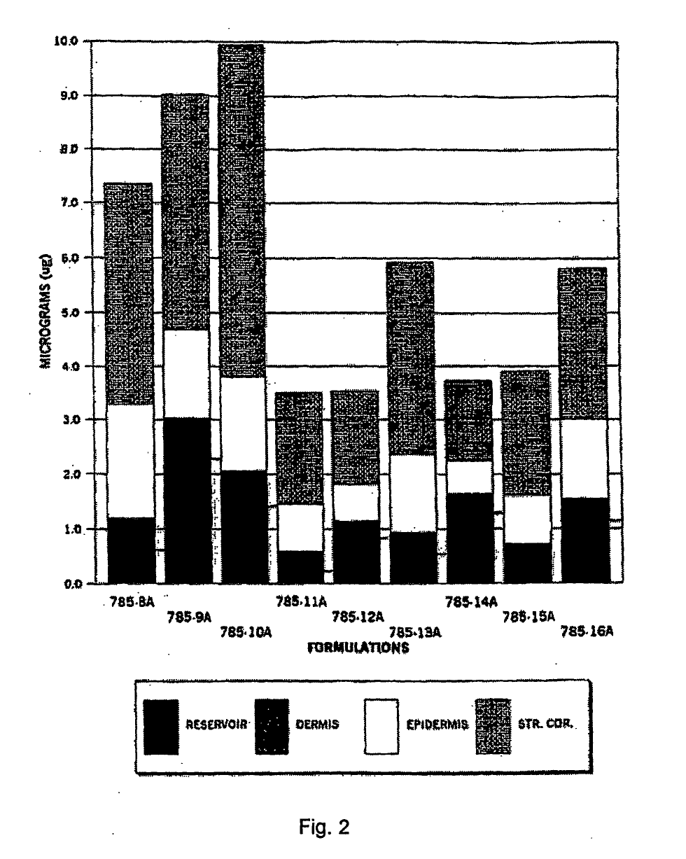

The cumulative transdermal absorption of alpha 1-antitypsin is presented as the percent of the applied dose recovered in the reservoir at each time point in FIG. 1. The skin specimens which showed ≧1.0% absorption in the reservoir compartment were removed from the reservoir data for the final data analysis due to apparent defect in barrier performance. The localization of alpha 1-antitrypsin in the various skin compartments is presented as both percent of the applied dose (DPM) recovered in each compartment as shown in FIG. 2.

All references cited herein are incorporated herein by reference in their entireties.

1. A composition for treating or preventing an inflammatory or hyperproliferative mucocutaneous disorder, comprising a protease inhibitor and a gelling agent.

2. The composition according to claim 1, wherein the protease inhibitor is an alpha 1-antitrypsin.

3. The composition according to claim 2, wherein the alpha 1-antitrypsin is a natural, synthetic or recombinant alpha 1-antitrypsin.

4. The composition according to claim 1, wherein the protease inhibitor is a modified peptide, biologically active fragment, substantially homologous polypeptide, oligopeptide, homodimer, heterodimer, variant, derivative, and/or an analog of alpha 1-antitrypsin.

5. The composition according to claim 1, further comprising a physiological buffer at a pH from about 6 to about 9.

6. The composition according to claim 5, wherein the buffer has a pH of from about 6.5 to about 7.5.

7. The composition according to claim 1, wherein the gelling agent is hydroxyethyl cellulose, hydroxypropyl cellulose, polyacrylic acid, a polyoxyethylene-polyoxypropylene block copolymer, or a combination thereof.

8. The composition according to claim 1, further comprising one or more pharmaceutically active agents.

9. The composition according to claim 1, which is sterile.

10. A pharmaceutical composition formulated for use in preventing or treating an inflammatory or hyperproliferative mucocutaneous disorder wherein said composition comprises a protease inhibitor and a gelling agent, and a pharmaceutical carrier.

11. The composition according to claim 10, wherein the inhibitor is alpha 1-antitrypsin.

12. The composition according to claim 10, wherein the composition further comprises one or more of the following:
a physiological buffer at a pH from about 6 to about 9;
a gelling agent that is hydroxyethyl cellulose, hydroxypropyl cellulose, polyacrylic acid, a polyoxyethylene-polyoxypropylene block copolymer, or a combination thereof; and/or
one or more pharmaceutically active agents.

13. (canceled)

14. (canceled)

15. (canceled)

16. A method of making a protease inhibitor gel composition, comprising:
(a) mixing a powdered gelling agent with an aqueous solution to form a gel;
(b) adjusting the pH of the gel to a pH of from about 5.5 to about 9.0;
(c) sterilizing the gel; and
(d) combining a protease inhibitor with the gel to form the protease inhibitor gel.

17. The method according to claim 16, wherein the aqueous solution is a physiological buffer.

18. The method according to claim 16, further comprising adjusting the pH of the protease inhibitor gel from about 5.5 to about 9.0.

19. The method according to claim 16, wherein the protease inhibitor is an alpha 1-antitrypsin.

20. The method according to claim 16, wherein the gelling agent is hydroxyethyl cellulose, hydroxypropyl cellulose, polyacrylic acid, polyoxyethylene-polyoxypropylene block copolymer, or a combination thereof.

21. The method according to claim 16, wherein the sterilizing comprises irradiation.

22. The method according to claim 16, further comprising lyophilizing the protease inhibitor gel.

23. A method for the treatment or prevention of an inflammatory or hyperproliferative mucocutaneous disorder, wherein said method comprises administering to a subject in need thereof an effective amount of a composition comprising a protease inhibitor and a gelling agent.

24. The method according to claim 23, wherein the protease inhibitor is an alpha-1 antitrypsin.

25. The method according to claim 23, wherein the composition further comprises a physiological buffer at a pH from about 6 to about 9.

26. The method according to claim 25, wherein the buffer has a pH of from about 6.5 to about 7.5.

27. The method according to claim 23, wherein the gelling agent is hydroxyethyl cellulose, hydroxypropyl cellulose, polyacrylic acid, polyoxyethylene-polyoxypropylene block copolymer, or a combination thereof.

28. The method according to claim 24, wherein the alpha 1-antitrypsin is a natural, synthetic or recombinant alpha 1-antitrypsin.

29. The method according to claim 23, wherein the composition further comprises one or more pharmaceutically active agents.

30. The method according to claim 23, wherein the disorder is a dermatological disorder, disorder of the ear, ocular disorder, disorder of the gastrointestinal tract, or disorder of the urinary tract.

31. The method according to claim 23, wherein the disorder is a dermatological disorder selected from the group consisting of atopic dermatitis; skin photodamage; extrinsic skin aging; skin irritation; chronic, burn and ulcer wounds; acne; psoriasis; lichen (particularly lichen planus); basal or squamous cell carcinoma (Bowen's disease); Kaposi's sarcoma; keratosis, such as actinic or seborrheic keratosis; and disorders of keratinization, such as ichthyosis (particularly lamellar ichthyosis) and keratoderma.

32. The method according to claim 23, wherein the disorder is otitis, conjunctivitis, colitis or intestinal cystitis.

33. The method according to claim 23, wherein the subject is a mammal.
